(19) United States
(12) Patent Application Publication
(10) Pub. No.: US 2018/0361357 A1
(43) Pub. Date: Dec. 20, 2018

(54) HYDROGENATION OR HYDROGENOLYSIS PROCESS

(71) Applicant: SHELL OIL COMPANY, HOUSTON, TX (US)

(72) Inventors: Dionysius Jacobus Maria DE VLIEGER, Amsterdam (NL); Jean Paul Andre Marie Joseph Ghislain LANGE, Amsterdam (NL); Hendrik Albertus COLIJN, Amsterdam (NL); Smita EDULJI, Houston, TX (US)

(21) Appl. No.: 16/062,139

(22) PCT Filed: Dec. 15, 2016
(86) PCT No.: PCT/EP2016/081270

§ 371 Date: Jun. 14, 2018

Related U.S. Application Data
(60) Provisional application No. 62/268,615, filed on Dec. 17, 2015.

(51) Int. Cl. B01J 21/06; B01J 23/28; B01J 23/30; B01J 23/42; B01J 23/44; B01J 23/46; B01J 23/72; B01J 23/745; B01J 23/75; B01J 23/755; B01J 37/02

(52) U.S. Cl. CPC B01J 21/063; C07C 29/132; B01J 23/28; B01J 23/30; B01J 23/42; B01J 23/44; B01J 23/462; B01J 23/464; B01J 23/468; B01J 23/72; B01J 23/745; B01J 23/75; B01J 23/755; B01J 37/0217; B01J 37/0225; B01J 21/066

(57) ABSTRACT

A catalytic process for the hydrogenation or hydrogenolysis of a reactant in a reactor in the presence of hydrogen and liquid water is disclosed. The catalyst is stable under hydrothermal conditions.

### FIELD OF THE INVENTION

The present invention relates to a process for the hydrogenation or hydrogenolysis of a reactant in a reactor in the presence of a catalyst, hydrogen and liquid water.

### BACKGROUND OF THE INVENTION

Supported catalysts, wherein a metal is dispersed on the surface of a support material such as a metal oxide, are used in many different chemical processes. The supported catalysts are prepared by well-known methods wherein the metal is deposited onto the support material.

Under hydrothermal conditions, wherein a process is carried out in the presence of water and at a high temperature, many commonly-used inorganic oxide catalyst supports are not stable. The catalyst supports may undergo phase changes or growth of crystallites, or may begin to dissolve. This can detrimentally affect catalyst performance, leading to lower product yield and a need to change the catalyst more frequently. This can also lead to system instability such that reaction conditions may need to be changed to maintain catalyst performance. Additionally, dissolution of catalyst supports can lead to the presence of impurities in the process.

Carbon catalyst supports might potentially be stable under hot, aqueous conditions but may also be mechanically fragile such that a portion of the catalyst is crushed when the supported catalyst is loaded into a reactor. Additionally, carbonaceous deposits may form on the catalysts, and a typical regeneration procedure of burning off the carbonaceous deposits would not be possible with a carbon catalyst support as the carbon support would also burn.

Duan et al in Catalysis Today 234 (2014), 66-74 discuss the use of titania and zirconia catalyst supports in the aqueous-phase hydrodeoxygenation of sorbitol. A catalyst is prepared by treating titania for 100 hours at 523K in the presence of liquid water to stabilise the material and then by adding platinum and rhenium to the support.

The present inventors have sought to prepare supported catalysts that are stable under hydrothermal conditions in hydrogenation or hydrogenolysis processes.

### SUMMARY OF THE INVENTION

Accordingly, the present invention provides a process for the hydrogenation or hydrogenolysis of a reactant in a reactor in the presence of a catalyst, hydrogen and liquid water, wherein the catalyst comprises at least one metal chosen from Groups 8 to 11 of the periodic table on a metal oxide support, and wherein the catalyst has been prepared by a process comprising steps of:
(a) heating the metal oxide support in liquid water to a temperature of at least 150° C. for a period of at least 2 hours to provide a treated support; and
(b) depositing at least one metal chosen from Groups 8 to 11 of the periodic table on the treated support.

The present inventors have found that by treating the metal oxide support, prior to the deposition of the catalytic metal onto the support, it is possible to provide a catalyst that is stable under the hydrothermal conditions of the process for hydrogenation or hydrogenolysis.

### DETAILED DESCRIPTION OF THE INVENTION

The present invention provides a process for the hydrogenation or hydrogenolysis of a reactant in a reactor in the presence of a catalyst, hydrogen and liquid water. In a hydrogenation reaction, hydrogen is added to a double or triple bond in a molecule. In a hydrogenolysis reaction, hydrogen cleaves a bond in a molecule. Suitably the reactant is an oxygenate (an organic compound that contains oxygen, e.g. an alcohol, an ether, an aldehyde or a ketone). Preferably the oxygenate is present in or derived from a saccharide-containing feedstock, and the process produces glycols. The saccharide-containing feedstock preferably comprises starch and/or compounds prepared by the hydrolysis of starch. Glucose may be prepared by the hydrolysis of starch or other methods and is another preferred component of the saccharide-containing feedstock. The saccharide-containing feedstock may also comprise one or more further saccharides selected from the group consisting of monosaccharides other than glucose, disaccharides, oligosaccharides and polysaccharides other than starch. Examples of polysaccharides other than starch include cellulose, hemicelluloses, glycogen, chitin and mixtures thereof.

The saccharide-containing feedstock may be derived from grains such as corn, wheat, millet, oats, rye, sorghum, barley or buckwheat, from rice, from pulses such as soybean, pea, chickpea or lentil, from bananas and/or from root vegetables such as potato, yam, sweet potato, cassava and sugar beet, or any combinations thereof. A preferred source of saccharide-containing feedstock is corn.

A glycols product stream resulting from the process is typically a mixture of glycols, wherein the main constituents are monoethylene glycol (MEG), monopropylene glycol (MPG) and 1,2-butanediol (1,2-BDO).

The temperature of the liquid water in the reactor is at least 80° C., suitably at least 130° C., preferably at least 160° C., more preferably at least 190° C. The temperature of the liquid water in the reactor is at most 300° C., suitably at most 280° C., preferably at most 270° C., more preferably at most 250° C. and most preferably at most 230° C. Preferably, the liquid water is heated to a temperature within these limits before addition of any starting material and is maintained at such a temperature as the reaction proceeds. Operating at higher temperatures has the potential disadvantage of increased amounts of side-reactions, leading to lower product yield.

The pH in the reactor is in the range of from 2.5 to 10, preferably from 3 to 7 and most preferably from 3.5 to 5. The preferred pH is suitably maintained by using a buffer. Suitable buffers will be known to the skilled person but include sodium acetate. The amount of buffer supplied to the reactor is suitably from 0.01 to 10 wt % of buffer based on the total weight of feedstock supplied to the reactor, preferably from 0.1 to 1 wt %. The preferred pH is a balance between reducing the amount of side reactions and maximising the yield (the inventors' investigations suggest that higher pH gives fewer side reactions but lower pH gives better catalyst activity).

The pressure in the reactor is suitably at least 1 MPa, preferably at least 2 MPa, more preferably at least 3 MPa. The pressure in the reactor is suitably at most 25 MPa, preferably at most 20 MPa, more preferably at most 18 MPa. Preferably, the reactor is pressurised to a pressure within these limits by addition of hydrogen before addition of any reactant or liquid water and is maintained at such a pressure as the reaction proceeds through on-going addition of hydrogen.

The process takes place in the presence of hydrogen. Preferably, the process takes place in the absence of air or oxygen. In order to achieve this in a batch process, it is preferable that the atmosphere in the reactor be evacuated and replaced an inert gas, such as nitrogen, and then with hydrogen repeatedly, after loading of any initial reactor contents, before the reaction starts. In order to achieve this in a continuous process, it is preferable that any inert gas is flushed out by maintaining hydrogen flow for a sufficient time.

The reactant is preferably supplied as an aqueous solution of the reactant in liquid water.

The catalyst has been prepared by a process comprising a first step of heating the metal oxide support in liquid water to a temperature of at least 150° C. for a period of at least 2 hours to provide a treated support. The metal oxide support is preferably heated to a temperature of at least 200° C. The metal oxide support is suitably heated to a temperature of less than 350° C., preferably less than 300° C. and more preferably less than 250° C. The metal oxide support is preferably heated for a period of less than 10 hours. The pressure is suitably at least the autogenous pressure, i.e. the steam saturation pressure at the operating temperature. The pressure may be higher if additional gas (e.g an inert, oxidising or reducing gas) is present. The pressure must be sufficiently high that at least some of the water is present as a liquid. The pH of the liquid water is suitably from 2.5 to 8, preferably from 2.5 to 7 and most preferably from 3 to 5.

The metal oxide support may be an oxide of a single metal but may also be a mixed metal oxide or a doped metal oxide. The metal oxide support is suitably chosen from oxides of metals in groups 4 and 5 of the periodic table or is ceria. Preferably the metal oxide support is an oxide of one or more of titanium, zirconium, cerium and niobium. Most preferably the metal oxide support is titania or zirconia.

In one embodiment of the invention, the metal oxide support is titania, optionally doped with up to 50 wt % of another element (based upon the weight of the metal oxide).

In another embodiment of the invention, the metal oxide support is zirconia, optionally doped with up to 50 wt % of another element (based upon the weight of the metal oxide).

In yet another embodiment of the invention, the metal oxide support is a mixed metal oxide comprising at least 10 wt % titania and at least 10 wt % zirconia (based upon the weight of the metal oxide).

The catalyst has been prepared by a process comprising a second step of depositing at least one metal chosen from Groups 8 to 11 of the periodic table on the treated support. Preferably the at least one metal is chosen from the group consisting of iron, cobalt, nickel, copper, ruthenium, rhodium, palladium, iridium and platinum. More preferably ruthenium is deposited upon the metal oxide support. If the metal is one or more noble metals (e.g. ruthenium, rhodium, palladium, iridium or platinum), then the amount of metal is suitably from 0.05 to 5 wt %, based on the weight of the metal oxide support, preferably from 0.1 to 2 wt %. If the metal is one or more base metals (e.g. iron, cobalt, nickel, copper), then the amount of metal is suitably from 1 to 80 wt %, based on the weight of the metal oxide support, preferably from 2 to 50 wt %, more preferably from 5 to 20 wt %.

At least one metal is deposited upon the treated support using methods known to the skilled person. Suitable methods include ion-exchange, impregnation (including continuously stirred impregnation and pore volume impregnation), deposition-precipitation and vapour deposition. Co-deposition may be used, particularly if the metal to be deposited is a base metal and a high metal loading (e.g. greater than 50 wt %) is targeted.

In one embodiment of the invention, a second catalyst is present in the reactor. The second active catalyst preferably comprises one or more homogeneous catalysts selected from tungsten or molybdenum, or compounds or complexes thereof. Most preferably, the second catalyst comprises one or more material selected from the list consisting of tungstic acid, molybdic acid, ammonium tungstate, ammonium metatungstate, ammonium paratungstate, tungstate compounds comprising at least one Group I or II element, metatungstate compounds comprising at least one Group I or II element, paratungstate compounds comprising at least one Group I or II element, heteropoly compounds of tungsten, heteropoly compounds of molybdenum, tungsten oxides, molybdenum oxides and combinations thereof. This catalyst is a retro-aldol catalyst, and in a preferred embodiment of the invention, the retro-aldol reaction and hydrogenation or hydrogenolysis take place in the same reactor. In other embodiments of the invention, a retro-aldol reaction may occur in a separate reactor prior to the hydrogenation or hydrogenolysis.

The residence time in the reactor is suitably at least 1 minute, preferably at least 2 minutes, more preferably at least 5 minutes. Suitably the residence time in the reactor is no more than 5 hours, preferably no more than 2 hours, more preferably no more than 1 hour.

The present invention is further illustrated in the following Examples.

### Procedure for Preparing Catalyst: Treatment of Support Materials

The support materials were treated in 250 ml Berghoff autoclaves with 200 ml inserts, which were filled with 150 ml of water. The pH of the water was adjusted to 3 by addition of acetic acid. The minimum amount of material used per test was 2 g.

The water was heated to 250° C. by placing the autoclaves in an oven. Under those conditions, an autogenous pressure of ˜40 bar was obtained in the autoclave. The catalyst support materials were separated from the water phase by cold filtration.

### Procedure for Preparing Catalyst: Deposition of Catalytic Metal

Ruthenium was deposited onto the catalyst supports using an incipient wetness method. The support was impregnated with an aqueous solution of Ru(NO)(NO₃)₃. The impregnated support was dried carefully and then calcined at 300° C. for 2 hours.

Activity of Hydrogenation Catalysts with Stable Supports

The hydrogenation activity of the catalysts was tested in a process for the hydrogenation of glycolaldehyde to ethylene glycol. 30 g of water, 0.3 g of glycolaldehyde and hydrogen (101 bar) were fed to the catalyst. The reactants were subjected to stirring at 1450 rpm and a temperature of 195° C. for 75 minutes.

Table 1 shows the different catalysts that were tested and table 2 shows the results of the hydrogenation reaction:

TABLE 1

| | | Amount | Heat |
|---|---|---|---|
| | Catalyst | (g) | treatment |
| Comparative | 1% Ruthenium | 0.045 | None |
| Example 1 | on SiO₂ | | |
| Comparative | Raney Ni 2800 | 0.012 | None |
| Example 2 | | | |
| Comparative | Raney Co 2724 | 0.015 | None |
| Example 3 | Ni Cr | | |
| | promoted | | |
| Example 1 | 0.4% Ru on | 0.113 | Support was |
| | Si-doped ZrO₂ | | treated for |
| | | | 70 hours in |
| | | | hot water |
| | | | (250° C., pH 3) |
| Example 2 | 0.3% Ru on Y- | 0.045 | Support was |
| | doped ZrO₂ | | treated for |
| | | | 70 hours in |
| | | | hot water |
| | | | (250° C., pH 3) |
| Example 3 | 0.3% Ru on Y- | 0.15 | Support was |
| | doped ZrO₂ | | treated for |
| | | | 70 hours in |
| | | | hot water |
| | | | (250° C., pH 3) |
| Example 4 | 0.4% Ru on | 0.113 | Support was |
| | TiO₂—ZrO₂ | | treated for |
| | | | 70 hours in |
| | | | hot water |
| | | | (250° C., pH 3) |

TABLE 2

| | Ethylene | Propylene | | 1,2- | |
|---|---|---|---|---|---|
| | Glycol | Glycol | HA | BDL | 1H₂BO |
| | (wt %) | (wt %) | (wt %) | (wt %) | (wt %) |
| Comparative | 84.4 | 0.0 | 0.0 | 0.0 | 0.0 |
| Example 1 | | | | | |
| Comparative | 74.6 | 0.0 | 0.8 | 2.8 | 4.1 |
| Example 2 | | | | | |
| Comparative | 100.3 | 0.0 | 0.0 | 0.0 | 0.0 |
| Example 3 | | | | | |
| Example 1 | 82.0 | 0.0 | 0.0 | 4.1 | 0.0 |
| Example 2 | 32.9 | 0.0 | 1.7 | 2.7 | 7.1 |
| Example 3 | 59.3 | 5.1 | 0.0 | 9.2 | 0.9 |
| Example 4 | 87.8 | 0.0 | 0.0 | 4.8 | 0.0 |

The examples show that good activity can be achieved with the catalysts produced by the process of the invention.

1. A process for the hydrogenation or hydrogenolysis of a reactant in a reactor in the presence of a catalyst, hydrogen and liquid water, wherein the catalyst comprises at least one metal chosen from Groups 8 to 11 of the periodic table on a metal oxide support, and wherein the catalyst has been prepared by a process comprising steps of:
(a) heating the metal oxide support in liquid water to a temperature of at least 150° C. for a period of at least 2 hours to provide a treated support; and
(b) depositing at least one metal chosen from Groups 8 to 11 of the periodic table on the treated support.

2. The process according to claim 1, wherein the metal oxide support is titania, optionally doped with up to 50 wt % of another element; the metal oxide support is zirconia, optionally doped with up to 50 wt % of another element; or the metal oxide support is a mixed metal oxide comprising at least 10 wt % titania and at least 10 wt % zirconia.

3. The process according to claim 1, wherein the at least one metal chosen from Groups 8 to 11 of the periodic table is chosen from the group consisting of iron, cobalt, nickel, copper, ruthenium, rhodium, palladium, iridium and platinum.

4. The process according to claim 1, wherein the reactant is an oxygenate.

5. The process according to claim 4, wherein the oxygenate is present in or derived from a saccharide-containing feedstock, and the process produces glycols.

6. The process according to claim 1, wherein the temperature in the reactor is at least 190° C. and at most 250° C.

7. The process according to claim 1, wherein a second catalyst is present in the reactor and the second catalyst comprises one or more homogeneous catalysts selected from tungsten or molybdenum, or compounds or complexes thereof.
